(19) United States
(12) Patent Application Publication
(10) Pub. No.: US 2013/0325097 A1
(43) Pub. Date: Dec. 5, 2013

(54) WOUND DRESSING FOR ELECTRICAL STIMULATION, AND METHOD FOR PRODUCING SUCH A WOUND DRESSING

(71) Applicant: Hagen Loest, Norderstedt (DE)

(72) Inventor: Hagen Loest, Norderstedt (DE)

(21) Appl. No.: 13/991,363

(22) PCT Filed: Dec. 5, 2011
(86) PCT No.: PCT/EP2011/071787

§ 371 Date: Aug. 14, 2013

(30) Foreign Application Priority Data
Dec. 3, 2010 (DE) 102010053213.4

(51) Int. Cl. A61N 1/04

(52) U.S. Cl. CPC A61N 1/0472
USPC 607/149; 29/825

(57) ABSTRACT

The invention relates to a wound dressing (10) for electrical stimulation, with a planar energy-transmitting element (12, 12′), a protecting and supporting layer (14), which is connected to the energy-transmitting element (12, 12′) and which covers the energy-transmitting element (12, 12′) on the outside and, at least in some regions, protrudes beyond an edge (16, 16′) of the energy-transmitting element (12, 12′), and a planar wound cover (26) which is applied internally to a side of the energy-transmitting element (12, 12′) facing away from the protecting and supporting layer (14), wherein the energy-transmitting element (12, 12′) and the protecting and supporting layer (14) have a separating line (18, 18′), which extends from the edge into the interior of the wound dressing (10) and which separates a contact band (20, 20′) of the energy-transmitting element (12, 12′); from a wound covering and delimiting area (22, 24) of the energy-transmitting element (12, 12′). Moreover, the invention also relates to a method for producing such a wound dressing (10).

The invention relates to a wound dressing for electrical stimulation with a planar energy-transmitting element, a protecting and supporting layer which is connected to the energy-transmitting element, which covers the energy-transmitting element on the outside and, at least in some areas, protrudes beyond a periphery of the energy-transmitting element, and a planar wound cover which is applied internally to a side of the energy-transmitting element facing away from the protecting and supporting layer.

Furthermore, the invention relates to a method for producing such a wound dressing and a device for electrical stimulation of a wound, having such a wound dressing.

Such wound dressings for electrical stimulation as part of electrical stimulation devices are known from the prior art and are used for the treatment of wounds, especially of poorly healing, chronic wounds.

In EP 0 504 715 A1 a wound treatment device is described which comprises an electrically non-conductive layer, a non-metallic but electrically conductive layer and a substantially non-adhering wound contact layer. An approximately centrally arranged connection tab of the electrically conductive layer extends through an aperture of the electrically non-conductive layer to the outside, so that the electrically-conductive layer may be connected via this connection tab to a current source.

The generic WO 20041069088 A2 also discloses a wound dressing for electrical stimulation, in which an energy-transmitting element has a multilayer structure and comprises at least a first layer for energy supply as well as an adjacent second layer for energy distribution, wherein the first layer has a lower electrical resistance than the second layer. This leads to an extremely homogeneous current distribution over the entire surface of the energy-transmitting element, whereby a particularly uniform electrical stimulation of the wound and thus a substantially improved wound healing method can be achieved. Due to the improved energy distribution in the multilayer structure of the energy-transmitting element, embodiments of the wound dressing, in which the electrical connection of the energy-transmitting element is arranged peripherally, are already disclosed in this publication. Thereby the originally centred, that is, arranged in the centre of the wound and thus disruptive current connection can be cancelled for improved current distribution.

The embodiment of the wound dressing for electrical stimulation with a current connection arranged peripherally illustrated in WO 2004/069088 A2, however, has a less compact form. Due to this form there is also an undesirably high wastage of the energy-transmitting element. Moreover, it would be desirable for the wound dressing protecting the wound to be adhered circumferentially around the entire wound covering or wound on a patient's skin, in particular also in the area of the current connection.

The object of the invention is therefore to provide a particularly compact wound dressing for electrical stimulation, in which there is little wastage particularly in relation to the energy-transmitting element, and which is able to be produced at minimal cost.

This object is achieved by a wound dressing for electrical stimulation of the aforementioned type, wherein the energy-transmitting element and the protecting and supporting layer have a separating line which extends from the periphery into the interior of the wound dressing and which separates a contact band of the energy-transmitting element from a wound covering and delimiting area of the energy-transmitting element.

The length of the separating line is advantageously chosen so that a conducting connection between the contact band of the energy-transmitting element and the wound covering and delimiting area of the energy-transmitting element remains. In other words, the wound dressing is not then severed along its whole length by the separating line. The contact band severed by the separating line can then be used advantageously to connect the wound dressing to a device for electrical stimulation. The separating line initially facilitates a simple production of the wound dressing in compact form and with little material wastage. Subsequently, however, this separating line also facilitates, before any use of the wound dressing for electrical stimulation, that is, before its application to a wound, a flip or a fold of the contact band, so that this contact band protrudes laterally from the rest of the wound dressing and the wound dressing for electrical stimulation by the contact band can be connected easily to the current source. Advantageously, the energy-transmitting element in the area of the contact band in the direction from the periphery towards the interior of the wound dressing is dominated by the protecting and supporting layer in a first dimension.

The protecting and supporting layer is made shorter than the energy-transmitting element in a second dimension (i.e. perpendicular to the first dimension) in the contact band. The protecting and supporting layer, particularly along the periphery (i.e. along the contact band on the outer periphery of the wound dressing) to the corner of the wound dressing, is shorter than the energy-transmitting element. As a result, the energy-transmitting element extends further to the periphery of the wound dressing beyond the protecting and supporting layer surmounting the energy-transmitting element.

A corner of the protecting and supporting layer may be separated advantageously by a rectangular section or segment. The rectangular section or segment can then be at least as wide as the contact band. The separated corner can advantageously have a length, through which the projection of the energy-transmitting element (i.e. the layer) is then defined, if the protecting and supporting layer is folded over the energy-transmitting element.

The protecting and supporting layer is advantageously electrically insulated. Due to the dimensioning of the protecting and supporting layer and the layer of the energy-transmitting element, it is possible to separate only at least one corner of the protecting and supporting layer and so to enable access by means of the contact band on the energy-transmitting element if the protecting and supporting layer is folded on the energy-transmitting element.

In one embodiment of the wound dressing for electrical stimulation the planar energy-transmitting element is constructed in at least two layers, wherein a first layer for the energy supply is adjacent to the protecting and supporting layer and a second layer for energy distribution is adjacent to the wound covering.

Here, the first layer preferably has a lower electrical resistance than a second layer. This structure of the energy-transmitting element results in a very homogeneous energy distribution and electrical stimulation over the entire wound surface, whereby the wound healing method is significantly improved. Due to this improved energy distribution the current connection of the wound dressing may also take place at any point without the wound healing method being impaired.

In a further embodiment of the wound dressing for electrical stimulation a delimiting strip is applied to the delimiting area of the energy-transmitting element between the wound dressing and separating line. This delimiting strip separates the wound covering area from the contact band and is preferably made of a similar, more preferably identical, material to the protecting and supporting layer.

In this embodiment the protecting and supporting layer and the delimiting strip may form an adhesive periphery surrounding the wound covering for fixing the wound dressing. Since the adhesive periphery completely surrounds the wound covering and therefore, in case of use, the wound, the wound covering or wound is completely sealed from the environment and thus particularly reliably protected from contamination.

As an adhesive periphery a circumferential or planar covering adhesive layer may be applied to the protecting and supporting layer. This may be advantageously suitable both to connect further layers (preferably centrally located in the centre) adhering to the supporting layer as well as to adhere to the skin.

Advantageously, the protecting and supporting layer is vapour permeable. Thus maceration of the skin is avoided. Also advantageously, the adhesive periphery on the protecting and supporting layer is designed then as a liquid. A polyurethane foam, for example, is considered as the material for the protecting and supporting layer. Other materials may also be used.

Preferably, the circumferential adhesive periphery has a substantially constant width. The wound dressing can thus adhere well and offers a circumferentially uniform protection against contamination.

The separating line is preferably formed by perforation, cutting or punching of the energy-transmitting element and the protecting and supporting layer. Such an embodiment of the separating line allows a straightforward conversion of the wound dressing from a compact form of production into a form for use facilitating the electrical connection. If the separating line is pre-cut or pre-punched, then the contact band should only be flipped or folded in order to transform the wound dressing from the form of production to the form for use. If the separating line, however, is a linear perforation, then the contact band is initially fixed in the compact form of production. Preferably during the production of the wound dressing, the contact band is separated along the linear perforation without further aids and subsequently flipped or folded.

In a further embodiment of the wound dressing for electrical stimulation the planar energy-transmitting element and the protecting and supporting layer are formed substantially rectangular. In other embodiments of the invention, the planar energy-transmitting element and/or the protecting and supporting layer may also be formed oval, round or triangular. They are considered in conjunction with the various advantageous aspects of the invention variably and in various forms other than those mentioned.

The separating line in this embodiment preferably extends substantially parallel to a periphery of the planar energy-transmitting element. Consequently, only a simple, straight separating line is necessary to separate a strip of the energy-transmitting element and the protecting and supporting layer from the rest of the wound dressing, wherein said strip forms the contact band during operation of the wound dressing for electrical stimulation.

In a further embodiment the protecting and supporting layer adjacent to the contact band of the energy-transmitting element comprises a protruding peripheral area, wherein a width of this peripheral area corresponds substantially to a width of the contact band. The peripheral area of the protecting and supporting layer can thus fold over the contact band of the energy-transmitting element so that the contact band is protected on both sides by the protecting and supporting layer and so that the protecting and supporting layer is also electrically insulated using a suitable material. Since the peripheral area folded over the contact band is usually coated with an adhesive on the side facing the contact band, this peripheral area of the protecting and supporting layer is also advantageously fixed to the contact band during folding.

A first contact band end is preferably connected to the wound covering area of the energy-transmitting element and an exposed, second contact band end is able to be connected to a current source.

Here, the energy-transmitting element and the protecting and supporting layer may be flush with one of the lateral peripheries of the second contact band end opposite the separating line. Thus, the contact band of the energy-transmitting element is not completely covered during folding of a peripheral area of the protecting and supporting layer. At the second contact band end the energy-transmitting element is exposed and advantageously forms a contact surface for connection of the wound dressing for electrical stimulation to a current source.

In order to avoid undesirable, protruding peripheral sections of the protecting and supporting layer at the second contact band end, the energy-transmitting element and the protecting and supporting layer are also preferably flush with an end face of the second contact band end.

According to one aspect of the invention, the wound dressing is heat-generating during energisation.

The wound dressing can have advantageous substances promoting wound healing.

A substance promoting wound healing can be a growth factor.

Substances promoting wound healing may for example be chemically or pharmaceutically active substances. In particular for this purpose are antibiotics, antiseptics, vitamins, analgesics, collagens, hydrocolloids, alginates, foams or other active substances.

The invention also comprises a device for electrical stimulation of a wound, using a previously described wound dressing for electrical stimulation, a current source for supplying current to the wound dressing and an electrical line for connecting the current source to an exposed end of the contact band.

The object assigned, moreover, is also achieved by a method of producing a wound dressing for electrical stimulation with the following steps:
provision of a protecting and supporting layer;
application of the energy-transmitting element to the protecting and supporting layer,
application of a layer of gel;
application of an insulating strip to the energy-transmitting element;
application of a separating line by perforating, punching or cutting of the energy-transmitting element and/or the protecting and supporting layer;
separation of at least one corner of the protecting and supporting layer;
folding of the protecting and supporting layer over the energy-transmitting element;
cutting of the protecting and supporting layer; and
application of the wound dressing to a transport supporting layer.

The corner of the protecting and supporting layer may advantageously be separated by a rectangular section or segment. The rectangular section or segment may then be at least as wide as the contact band. The separated corner may advantageously have a length, through which the projection of the energy-transmitting element (i.e. the layer) is then defined, if the protecting and supporting layer is folded over the energy-transmitting element.

In particular, a sterilisation of the wound dressing may be performed. The application to the transport supporting layer protects the wound dressing from drying out and keeps it sterile. Using silicone adhesive a gas sterilisation, in particular, and subsequent further packaging steps may be advantageous.

The production method according to the invention thereby provides above all the possibility to carry out the production continuously, based on lengths of the protecting and supporting layer, the energy-transmitting element and other layers.

In this production method a separating line is advantageously applied, through which a compact shape of the wound dressing can be maintained initially. At the same time this separation line, however, also allows for a simple change in shape of the wound dressing for electrical stimulation before use, wherein this change in shape makes the connection of the wound dressing to a current source easier.

In particular, during the production method according to the present invention the wound dressings may be continuously separated from each other at the ends. This is the case even, for example, if the packaging has already also been closed around the individual wound dressings as a layer.

Further advantageous embodiments of the inventive concept are described in the subclaims.

Additional features and advantages of the invention will become apparent from the following description of preferred embodiments with reference to the drawings. In the drawings:

FIG. 1 shows a plan view of a planar energy-transmitting element and a protecting and supporting layer of a wound dressing according to the invention according to a first embodiment;

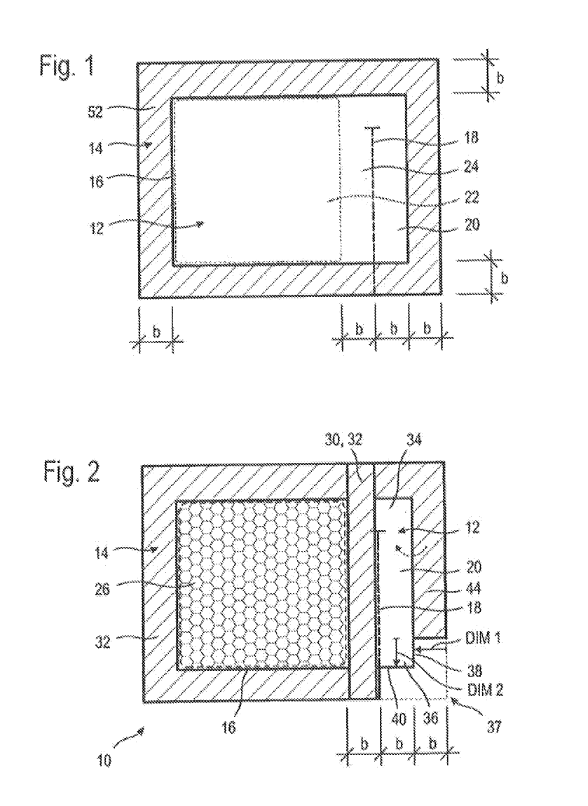

FIG. 2 shows a plan view of a wound dressing according to the invention according to the first embodiment;

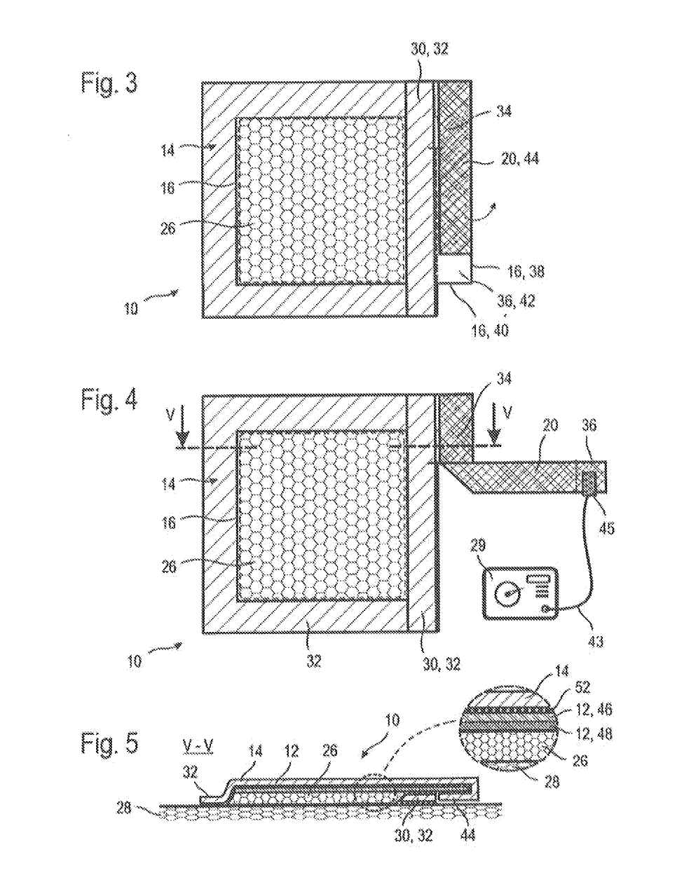

FIG. 3 shows a further plan view of the wound dressing according to the first embodiment;

FIG. 4 shows a further plan view of the wound dressing according to the first embodiment;

FIG. 5 shows a cutting V-V through the wound dressing for electrical stimulation according to FIG. 4;

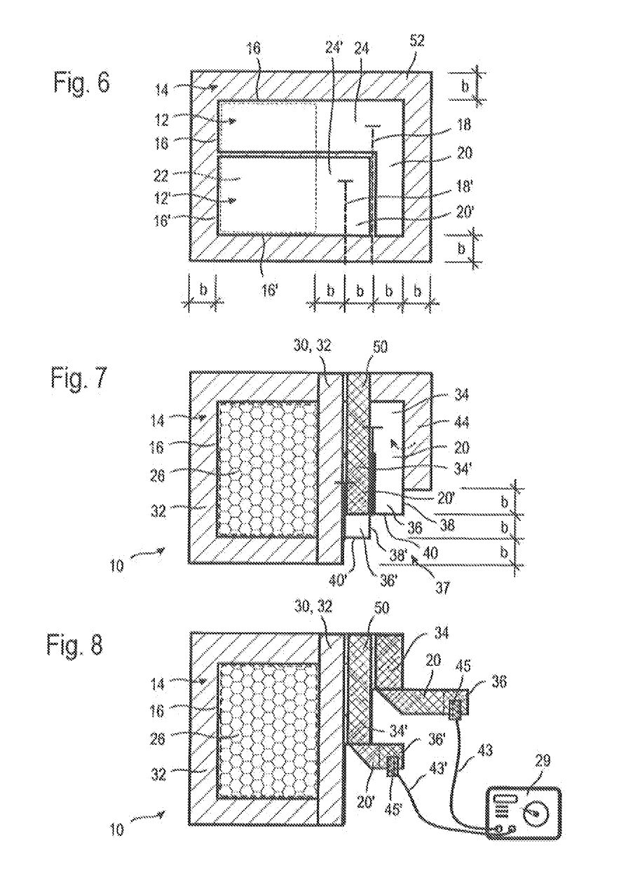

FIG. 6 shows a plan view of planar energy-transmitting elements and a protecting and supporting layer of a wound dressing according to the invention according to a second embodiment;

FIG. 7 shows a plan view of a wound dressing according to the invention according to the second embodiment; and

FIG. 8 shows a further plan view of the wound dressing according to the second embodiment.

FIGS. 1 to 4 show various steps of a method for producing a wound dressing for electrical stimulation 10 according to a first embodiment.

According to FIG. 1 a planar energy-transmitting element 12 and a protection and supporting layer 14 are initially provided. The energy-transmitting element 12 is in this case adhesively bonded to the protecting and supporting layer 14, so that the protecting and supporting layer 14 covers the energy-transmitting element 12 on the outside, adheres to the energy-transmitting element 12 and protrudes at least in sections over a periphery of the energy-transmitting element 12.

The protecting and supporting layer 14, after the application of the wound dressing 10 to a wound, is the outermost layer and is used in addition to fixing also for protecting the wound dressing 10 and the wound. Typically, the protecting and supporting layer 14 is produced from a film-like plastic material or a tissue. In principle, any material which is electrically non-conductive and is used in conventional wound dressings or bandages as a protecting and supporting layer is suitable as a protecting and supporting layer 14. Typically, the protecting and supporting layer 14 is coated on the interior with a skin-compatible adhesive, so that the wound dressing 10 can be attached easily to the skin 28 of a patient (see FIG. 5). Suitable adhesives are well known from bandage production.

The planar energy-transmitting element 12 is used for energy supply and energy distribution, so that the wound undergoes a uniform electrical stimulation. A uniform electrical stimulation is preferably then achieved when the electric field of the wound covering generated over the entire wound surface is homogeneous. The energy-transmitting element 12 is a film-like element, preferably a film-like composite element having at least two layers, i.e. at least two planar overlapping film-like elements. Further advantageous details of the energy-transmitting element 12 appear in FIG. 5. Furthermore, with regard to advantageous embodiments of the energy-transmitting element 12, explicit reference is made to WO 2004/069088 A2.

FIG. 1 shows the plan view of an interior of the energy-transmitting element 12 and the protecting and supporting layer 14. The interior in each case is understood to be that side which faces the skin 28 or the wound during use of the wound dressing 40. A circumferential or planar adhesive or bonding layer 52 is on the protecting and supporting layer. This can be used as a standalone layer or as part of the protecting and supporting layer.

A separating line 18, which has been applied by perforating, punching or cutting the energy-transmitting element 12 and the protecting and supporting layer 14, is also already evident in FIG. 1. This separating line 18 separates a contact band 20 of the energy-transmitting element 12 from the rest of the energy-transmitting element 12, wherein the rest of the energy-transmitting element 12 is divided into a wound covering area 22 and a delimiting area 24. The adhesive or bonding layer 52 on the protecting and supporting layer is advantageously designed such that the layer of the energy-transmitting element 12 adheres well. The adhesive or bonding layer 52 (it may also be designed as an adhesive surface of the supporting layer 14) also adheres uniformly and without side effects (or with few side effects) to the skin of the patient.

In the present case, the planar energy-transmitting element 12 and the protecting and supporting layer 14 are formed substantially rectangular, and the separating line 18 extends substantially parallel to the periphery 16 of the energy-transmitting element 12.

According to FIG. 2 a planar wound covering 26 has been applied inside the wound covering area 22 of the energy-transmitting element 12. This wound covering 26, in the present exemplary embodiment, is a gel or other material which may be hydrophilic and is electrically conductive, and may contain substances promoting wound healing. In particular, foams are also considered instead of a gel. These foams can then be provided advantageously with substances or soaked in liquid substances. When the wound dressing 10 is in use the wound covering 26 (for example, the gel or foam or the like) is in direct contact with the wound or skin 28. Before the wound dressing 10 is applied to the wound, however, a (not shown) film or transport supporting layer, which is arranged on the surface of the wound covering 26 (gel, foam, etc.), is removed and thus protects the wound covering from contamination.

It is important, however, that an easy removal of the film or transport supporting layer is possible without removing portions of the wound covering 26. So that the wound dressing 10, in particular the wound covering (for example, gel), does not lose liquidity or substances promoting wound healing during storage, the film or transport supporting layer is designed to be waterproof and resistant to active substances. Moreover, the film or transport supporting layer extends preferably not only over the wound covering 26 but over the entire inside of the protecting and supporting layer 14.

After the film or transport supporting layer is removed, the wound dressing 10 is applied to the wound to be treated. The wound covering (for example, gel or foam) in this case has adhesive properties on its surface so that it adheres effectively to the skin 28 or wound—even over long periods of time. The adhesive, which also serves to allow the wound dressing 10 to adhere to the skin of the patient, also serves equally advantageously to attach the wound dressing 10 to the transport supporting layer.

During the stimulation treatment the wound dressing 10 is supplied with current, in particular with current pulses. In this case, current flows from a current source 29 (FIG. 4) via the contact band 20 into the energy-transmitting element 12 formed as a planar electrode. From there, the electrical stimulation is transmitted to the wound through the underlying wound covering 26 (for example, gel/foam, etc.), which has a homogeneous electrical conductivity, whereby in the illustrated embodiment, a uniform stimulation, preferably a homogeneous electric field, may be obtained over the whole wound area.

During the stimulation treatment the wound covering (for example, the gel/foam, etc.) may deliver wound healing active substances to the wound, which can speed up the healing process. Simultaneously, the wound covering 26 is to absorb wound exudate in greater quantities due to its composition. In this case, the absorption capacity of liquid (such as water or wound exudate) is ensured by the absorber. Polymers, for example, are also suitable as absorbers. Moreover, the wound covering (for example, the gel/foam) is structurally stable, particularly during the stimulation treatment of the wound and while the wound remains.

According to FIG. 2 a delimiting strip 30 has also been applied internally to the delimiting area 24 of the energy-transmitting element 12, which separates the wound covering area 22 of the energy-transmitting element 12 from the contact band 20 of the energy-transmitting element 12.

In the illustrated interior plan view the protecting and supporting layer 14 and the delimiting strip 30 form an adhesive periphery 32 surrounding the wound covering 26 for fixing the wound dressing 10. After application of the wound dressing 10 to the skin 28, the wound covering 26 and the wound are thus separated by the circumferential adhesive bond from the environment and thus protected from contamination. To ensure a circumferentially uniform protection the adhesive periphery 32 has a substantially constant width b (see also FIG. 1).

The delimiting strip 30 in the present exemplary embodiment is made of the same material as the protecting and supporting layer 14. This also means that it is coated internally with a skin-compatible adhesive. To attach the delimiting strip 30 to the energy-transmitting element 12, this is also coated on the outside with an adhesive. Although the outside of the delimiting strip 30 does not come into contact with the skin 28, the adhesive used is preferably identical to the adhesive applied internally for reasons of simpler wound dressing production.

The contact band 20 has a first contact band end 34 which is connected to the wound covering area 22 of the energy-transmitting element 12, and an exposed, second contact band end 36, which is able to be connected to the current source 29.

The planar energy-transmitting element 12 and the protecting and supporting layer 14 are formed substantially rectangular according to FIG. 1 and the protecting and supporting layer 14 initially protrudes circumferentially beyond the periphery 16 of the energy-transmitting element 12. In addition to a rectangular shape, oval, round and/or triangular or other configurations are also considered. It has been assumed that the protecting and supporting layer 14 according to FIG. 2 is cut to a step-shape in a corner area 37. This step-shaped incision originates through the above-mentioned step of the production method, in which at least one corner of the protecting and supporting layer (14) is separated. In alternative embodiments, the incision may extend into the energy-transmitting element 12, so that both the protecting and supporting layer 14 and the energy-transmitting element 12 are incised in the corner area 37.

The step-shaped incision in the corner area 37 is preferably formed so that the energy-transmitting element 42 and the protecting and supporting layer 14 are flush with one of the lateral peripheries 38 of the second contact band end 36 opposite the separating line 48.

Due to the stepped shape of the incision the energy-transmitting element 12 and the protecting and supporting layer 14, moreover, are also flush with an end face 40 of the second contact band end 36.

The incision described subsequently allows for straightforward connection of the second contact band end 36 to the current source 29 without interfering sections of the protecting and supporting layer 14 bonding to or covering a contact surface 42 on the second contact band end 36.

FIG. 2 also now illustrates the aforementioned first direction DIM1 and second direction DIM2. To allow access to the contact band at least one corner of the protecting and supporting layer 14 in the corner area 37 must be removed. Moreover, the protecting and supporting layer 14, as indicated by the arrow DIMI, should here protrude beyond the layer of the energy-transmitting element 12 (along the arrow DIMI). Similarly, the layer of the energy-transmitting element 12 should protrude beyond the protecting and supporting layer 14 along the arrow DIM2 relative to the outer edge. The first direction DIM1 is then perpendicular to the second direction DIM2. The protecting and supporting layer 14 protrudes beyond the layer of the energy-transmitting element 12 in the first direction (i.e. overlaps the layer of the energy transmitting element) and at least one corner of the layer of the energy-transmitting element 12 protrudes in a second direction disposed perpendicularly thereto, i.e. the layer of the energy-transmitting element 12 is longer than the protecting and supporting layer 14 in the second direction DIM2. Now at least one corner of the protecting and supporting layer 14 (the stepped shape is not absolutely necessary) is separated, so that the contact band 34 from the layer of the energy-transmitting element 12 can be covered by folding the protruding protecting and supporting layer 14. Thus, a double-sided insulated contact band may be created which has at one end an exposed contact band end 36.

The significance of this incision is clear in view of FIG. 3, in which one of the protruding periphery areas 44 of the protecting and supporting layer 14 adjacent to the contact band 20 has been folded over, based on FIG. 2, so that the periphery area 44 covers an interior of the contact band 20 (with the exception of the second contact band end 36). As a result of the step-shaped incision the energy-transmitting element 12, however, is still exposed on the second contact band end 36 and forms the contact surface 42, which can be connected, for example, via an electrical connection line 43 to the current source 29 (see FIG. 4). Because of the exposed energy-transmitting element 12 in the area of the contact surface 42, the electrical connection between the exposed, second contact band end 36 and the connecting line 43 can be produced easily, for example by a contact terminal 45 disposed at one end of the connection line 43.

In order that the contact band 20 may be covered over its entire width b, a width of the periphery area 44 corresponds substantially to the width b of the contact band 20. In addition, since the interior of the periphery area 44 is coated with an adhesive, the periphery area 44 remains fixed in the folded position shown in FIG. 3 and the contact band 20, with the exception of its second contact band end 36, is sufficiently electrically insulated due to the double-sided covering with the electrically non-conductive protecting and supporting layer 14.

In an inventive method of producing a wound dressing for electrical stimulation, therefore, a strip from a protecting and supporting layer 14 may then be provided. From this, by means of a strip, the energy-transmitting element 12, 12′ (strip-like) may also be applied to the protecting and supporting layer 14. Thereupon a gel layer may also be applied. Subsequently, an insulating strip 32 may be applied to the energy-transmitting element 12, 12′. Then a separating line 18, 18′ may be applied by perforating, punching or cutting the energy-transmitting element 12, 12′ and/or the protecting and supporting layer 14. Subsequently, at least one corner of the protecting and supporting layer 14 must be separated (corner 37, as shown in FIG. 2). Then in an automated process the protecting and supporting layer 14 may also be folded over the energy-transmitting element 12, 12′, so that the contact band is covered, except for one end section 36. The separating line is of course advantageously designed in such a way that it does not sever the energy-transmitting element completely, as otherwise a supply of energy to the energy-transmitting element through the contact band would no longer be possible.

Subsequently, the protecting and supporting layer 14 may be cut and the wound dressing may be applied to a transport supporting layer (film, etc.). The protecting and supporting layer advantageously has an adhesive with which the wound dressing may be applied adhesively to the skin of a patient. This adhesive is advantageously composed so that by using it the application of the wound dressing to the transport supporting layer (film) is also possible.

FIG. 4 shows the plan view of an interior of the wound dressing for electrical stimulation 10 in its form of use, in which the contact band 20 has been folded over so that it extends away from the wound covering 26 and thus facilitates a simple electrical connection to the current source 29. So that the condition of the patient is not affected by interfering contact terminals 45 and simultaneously so that the connection of the wound dressing for electrical stimulation 10 to the current source 29 is possible with a simple manipulation, it has been found to be particularly advantageous if the distance between the second contact band end 36 and the wound covering 26 is approximately 4 cm.

FIG. 5 shows a cutting V-V through the wound dressing 10 according to FIG. 4, wherein the wound dressing 10 is applied to the skin of a patient 28. As a result the thickness of the individual layers is exaggerated for reasons shown in the drawings, which is why the cutting is only to be seen schematically.

Clear from a detail of the cutting V-V is that the planar energy-transmitting element 12 is constructed in two layers, wherein a first layer 46 of the energy supply system is used and is adjacent to the adhesive layer 52 of the protecting and supporting layer 14, and wherein a second layer 48 of the energy distribution is used and is adjacent to the wound covering 26. Now in order to maintain a uniform electrical stimulation over the whole contact surface of the wound covering 26, the first layer 46 has a lower electrical resistance to the energy supply than the second layer 48 has to the energy distribution. A uniform electrical stimulation is preferably then achieved if the electric field generated by the wound covering is homogeneous over the whole wound surface. The energy-transmitting element 12 is thus a multi-layered electrode with a low-impedance first layer 46 and a higher impedance second layer 48 (i.e. with a higher specific resistance than the low-impedance layer).

In particular the first layer 46 can be a layer of silver, whereby a lower electrical resistance is achieved. The incoming current is homogeneously distributed over the entire surface of the energy transmitting element 12 in this layer of silver due to its good electrical conductivity and then reaches the wound covering 26 via the second layer 48. The layer of silver therefore constitutes an effective medium for the wound dressing 10 in order achieve a uniform stimulation treatment, in particular a uniform or h electric field, over the entire wound surface of the wound to be supplied. In addition, using this layer of silver results in the possibility of arranging a current supply at any point in the energy-transmitting element 12, since an optimal current distribution is also ensured in a lateral disposition or, for example, in a corner of the energy-transmitting element 12. The insulating strip 32 may also be used as an adhesive or bonding layer. The strip 32 extends (right side) advantageously a little over the gel layer of the wound covering 26 and this thereby gives additional adhesion.

FIGS. 1 to 5 show that the separating line 18 provided for in the energy-transmitting element 12 and the protecting and supporting layer 14, which extends from the periphery of the wound dressing 10 to the interior of the wound dressing 10 and separates the contact band 20 of the energy-transmitting element 12 from a wound covering and delimitation area 22, 24 of the energy-transmitting element 12, has the positive effect of allowing on the one hand a compact form of production of the wound dressing 10 according to FIG. 2 or 3, but also of simultaneously enabling, with a single manipulation, a deformation of the wound dressing 10 in an advantageous form of use according to FIG. 4.

FIGS. 6 to 8, analogous to FIGS. 1 to 4, show plan views of the wound dressing for electrical stimulation 10 according to a second embodiment.

This second embodiment differs substantially in that there are two separate energy-transmitting elements 12, 12′, which are connected separately to the current source 29. In the present exemplary embodiment two energy-transmitting elements 12, 12′ are provided, arranged coplanar so that they lie side by side in a planar fashion. Alternatively, it is also conceivable that the energy-transmitting elements 12, 12′ overlap in terms of area, wherein they must be separated in the overlapping areas by means of an electrically insulated layer (not shown).

By means of this second embodiment with only one wound dressing 10 specific individual wound areas, i.e. preferably different to neighbouring wound areas, can be stimulated by current pulses. In some cases, this can lead to a further improved wound healing.

FIG. 6, analogous to FIG. 1, shows the protecting and supporting layer 14, to which two planar energy-transmitting elements 12, 12′ are applied. Also analogous to FIG. 1 a wound covering area 22 is defined, which in accordance with FIG. 6, however, is formed by both energy-transmitting elements 12, 12′. A delimiting area 24 is also provided, as well as two contact bands 20, 20′ and two separating lines 18, 18′.

FIG. 7, analogous to FIG. 2, shows the situation after application of the planar wound covering 26 and the delimiting strip 30. In addition, a cover strip 50 is provided, which covers and electrically insulates the contact band 20′. The second contact band end 36′ of the contact band 20′ is thereby not covered, to facilitate an easier connection to the current source 29, as already previously explained.

In contrast to the delimiting strip 30 the cover strip 50 is not coated on its interior with an adhesive, in order to avoid an unwanted adhesion of the contact band 20′ to the skin 28. Particularly preferred is a strip of material used as a cover strip 50, which also forms the protecting and supporting layer 14.

Also easily recognised in FIG. 7 is the step-shaped incision in the corner area 37 of the protecting and supporting layer 14, which in turn leads to the advantages, as they were already described on the basis of the first embodiment.

FIG. 8 shows the wound dressing 10 after a deformation of the form of production according to FIG. 7 in the form of use according to FIG. 8.

The previously described embodiments of the wound dressing for electrical stimulation 10 exhibit the fundamental characteristics shared in common that they are produced by a method, in which a compact, geometric shape (circle, triangle, rectangle, square) of a demonstrated planar energy-transmitting element 12, 12′ is separated along the separating line 18, 18′ into a wound covering and delimiting area 22, 24 as well as a contact band 20, 20′ for the current source. This method also involves a step where the peripheral area 44 of the protecting and supporting layer 14 is folded over and adhered to the contact band 20.

Another characteristic shared in common also lies in the procedural steps, so that the delimiting strip 30 is adhered to the delimiting area 24 of the energy-transmitting element 12, 12′.

Thus a wound dressing 10 which has a compact form of production and at the same time a connection-friendly form of use is formed in all embodiments. The packaging and delivery of the wound dressing 10 takes place in this case in the compact form of production in accordance with FIGS. 2 and 7. Alternatively, the peripheral area 44 may still be folded prior to the packaging and distribution of the wound dressing 10, so that FIG. 3 shows an additional, alternative example of a form of production. In any case, the transformation of the wound dressing 10 from its form of production (FIGS. 2, 3 and 7) to its connection-friendly form of use (FIGS. 4 and 8) occurs by a simple manipulation on the part of the end user. This must (depending on the form of production) optionally fold the peripheral area 44 and then in any case flip the contact band 20, 20′ so that the second contact band end 36, 36′ extends away from the wound covering 26. Depending on the embodiment of the separating line 18, 18′ as the line of an effected cutting, as the line of an effected punching or as the line of an effected perforation, the flipping of the contact band 20 takes place without resistance or tearing of the perforation effected as a line.

1. A wound dressing for electrical stimulation, the wound dressing comprising:
a planar energy-transmitting element having an outside and a periphery;
a protecting and supporting layer connected to the energy-transmitting element, the protecting and supporting layer covering the outside of the energy-transmitting element and protruding, at least in sections, beyond the periphery of the energy-transmitting element; and
a planar wound covering, which is applied inside a wound covering area and a delimiting area on a side of the energy-transmitting element facing away from the protecting and supporting layer, wherein the energy-transmitting element and the protecting and supporting layer have a separating line extending from the periphery into an interior of the wound dressing, the separating line separating a contact band of the energy transmitting element from the wound covering area and the delimiting area of the energy-transmitting element, wherein the length of the separating line is selected such that a conductive connection between the contact band of the energy-transmitting element and the wound covering area and the delimiting area of the energy-transmitting element is retained.

2. The wound dressing according to claim 1, wherein the energy transmitting element is surmounted by the protecting and supporting layer in an area of the contact band in a direction from the periphery towards the interior of the wound dressing.

3. The wound dressing according to claim 1, wherein the protecting and supporting layer is shorter than the energy-transmitting element in a direction along the outside of the contact band so that the energy-transmitting element extends beyond the protecting and supporting layer.

4. The wound dressing according to claim 1, wherein the protecting and supporting layer is an electrical insulator.

5. The wound dressing according to claim 1, wherein the energy-transmitting element comprises at least a first layer for supplying energy, the first layer being positioned adjacent to the protecting and supporting layer, and a second layer for energy distribution, the second layer being positioned adjacent to the wound covering.

6. The wound dressing according to claim 1, wherein the energy-transmitting element comprises at least a first layer for supplying energy and a second layer for current distribution, the first layer having a lower electrical resistance than the second layer.

7. The wound dressing according to claim 1, wherein a delimiting strip is applied to the delimiting area of the energy-transmitting element between the wound covering and the separating line.

8. The wound dressing according to claim 7, wherein the protecting and supporting layer and the delimiting strip form an adhesive periphery for fixing the wound dressing surrounding the wound covering.

9. The wound dressing according to claim 8, wherein the adhesive periphery has a substantially constant width.

10. A device for electrical stimulation of a wound, the device comprising a wound dressing according to claim 1, the device further comprising a current source for supplying current to the wound dressing and an electrical connection line for connecting the current source to an exposed end of the contact band.

11. A method for producing a wound dressing for electrical stimulation comprising:
providing a protecting and supporting layer;
applying an energy-transmitting element to the protecting and supporting layer;
applying a gel layer to the energy-transmitting element;
applying an insulating strip on the energy-transmitting element;
applying a separating line by perforating, punching or cutting the energy-transmitting element and/or the protecting and supporting layer;
separating at least one corner of the protecting and supporting layer from the energy transmitting element;
folding the protecting and supporting layer over the energy-transmitting element;
cutting the protecting and supporting layer; and
applying the wound dressing to a transport supporting layer.

12. The method according to claim 11, wherein a delimiting strip is applied to one of the sides of the energy-transmitting element facing away from the protecting and supporting layer, the delimiting strip separating a wound covering area of the energy-transmitting element from a contact band of the energy-transmitting element.

13. A method of producing a wound dressing for electrical stimulation, the method comprising:
providing a compact, basic geometric shape of a demonstrated planar energy-transmitting element;
dividing the energy-transmitting element along a separating line into a wound covering area and a delimiting area and a contact band for connecting the wound dressing to a current source.

14. The method according to claim 13, further comprising: folding a periphery area of a protecting and supporting layer over the contact band and adhering the protecting and supporting layer to the contact band.

15. The method according to claim 13, further comprising adhering a delimiting strip to a delimiting area of the energy-transmitting element.
